(19) United States
(12) Patent Application Publication
(10) Pub. No.: US 2008/0306155 A1
(43) Pub. Date: Dec. 11, 2008

(54) METHOD FOR TREATING RENAL DISEASE

(76) Inventors: Richard J. Roman, Brookfield, WI (US); John R. Falck, Dallas, TX (US)

Correspondence Address: QUARLES & BRADY LLP, 33 E. MAIN ST, SUITE 900, P.O BOX 2113, MADISON, WI, 53701-2113, US

(21) Appl. No.: 12/141,585

(22) Filed: Jun. 18, 2008

Related U.S. Application Data
(63) Continuation-in-part of application No. 11/453,132, filed on Jun. 14, 2006.
Application No. 11/453,132 is a continuation-in-part of application No. 11/229,241, filed on Sep. 16, 2005.
(60) Provisional application No. 60/610,465, filed on Sep. 16, 2004.

(51) Int. Cl. A61K 31/20; A61K 31/201; A61P 13/12; A61K 31/195

(52) U.S. Cl. 514/560; 514/563; 514/558

(57) ABSTRACT

A method preserving renal medullary blood flow in a renal disorder in a human or non-human animal is disclosed. The method involves administering 20-HETE or a 20-HETE analog to the human or non-human animal in an amount sufficient to attenuate a fall in renal medullary blood flow following a renal disorder. In addition, a method for preventing and treating ischemic acute renal failure is disclosed. The method involves administering 20-HETE or a 20-HETE agonist to the human or non-human animal in an amount sufficient to prevent or treat ischemic acute renal failure.

### CROSS-REFERENCE TO RELATED APPLICATIONS

This application is a continuation-in-part of U.S. patent application Ser. No. 11/453,132, which was filed on Jun. 14, 2006. U.S. patent application Ser. No. 11/453,132 is a continuation-in-part of U.S. patent application Ser. No. 11/229,241, which was filed on Sep. 16, 2005, and claims the benefit of U.S. Provisional Patent Application No. 60/610,465, which was filed on Sep. 16, 2004. The disclosure of each application is incorporated herein by reference as if set forth in its entirety.

### STATEMENT REGARDING FEDERALLY SPONSORED RESEARCH OR DEVELOPMENT

This invention was made with United States government support awarded by the following agency: NIH HL-36279. The United States government has certain rights in this invention.

### BACKGROUND

Diabetes and hypertension are leading causes of end-stage renal disease (ESRD). Despite effective medications, compliance and drug costs are serious problems, and only a small percentage of patients achieve adequate life-long control of their blood pressure or diabetes. Consequently, the incidence of ESRD is increasing as the population ages and becomes more obese. The cost to the US federal government for treating ESRD exceeds fifteen billion dollars a year.

Current treatment options for ESRD include dialysis and kidney transplant. These treatments, however, suffer from various drawbacks. For example, each is associated with high costs. In addition, dialysis provides only filtration but not other kidney functions; whereas kidney transplant can be plagued by organ shortage or rejection.

Recently, transforming growth factor-beta (TGF-•) was identified as a target for treating diabetes- and hypertension-induced nephropathies because its expression is upregulated in kidneys of patients and animal models with these diseases (Noble N & Border W, Sem. Nephrol. 17:455-466 (1997); Reeves W & Anderoli T, Proc. Natl. Acad. Sci. 97:7667-7669 (2000); Sharma K & McGowan T, Cytokine Growth Factor Rev. 11:115-123 (2000); Sharma K, et al, Diabetes 46:854-859 (1997); Yamamoto T, et al., Proc. Natl. Acad. Sci. 90:1814-1818 (1993); and Yamamoto T, et al., Kidney Intl. 49:461-469 (1996)). Diabetes- and hypertension-induced nephropathies are characterized by the early development of proteinuria, which accelerates the progression of renal disease by, e.g., promoting the development of glomerular lesions (e.g., glomerulosclerosis). TGF-overexpression can be a critical factor in this process (Dahly A, et al., Am. J. Physiol. Regul. Integr. Comp. Physiol. 283:R757-767 (2002); Border W, et al., N. Engl. J. Med. 331:1286-1292 (1994); Sanders P, Hypertension 43:142-146 (2004); McCarthy E, et al., J. Am. Soc. Nephrol. 14:84 A (2003); Bottinger E, et al., J. Am. Soc. Nephrol. 10:2600-2610 (2002); August P, et al., Kidney Intl. Suppl. 87:S99-104 (2003); and Ziyadeh F, et al., Proc. Natl. Acad. Sci. 97:8015-8020 (2000)).

For example, TGF-• directly increased the permeability of isolated glomeruli to albumin (Sharma R, et al., Kidney Intl. 58:131-136 (2000)), indicating a direct role of TGF-• in the induction of proteinuria. TGF-• also increased the production of extracellular matrix and promoted the development of glomerulosclerosis and renal interstitial fibrosis (Pavenstadt H, et al., Physiol. Rev. 83:253-307 (2003); Border et al., supra; and Sanders, supra). Importantly, blocking the activity of TGF-• by either TGF-• antibodies or antisense oligonucleotides reduced the degree of proteinuria and glomerular damage (Dahly et al., supra; Ziyadeh et al., supra; Chen S, et al., Biochem. Biophys. Res. Commun. 300:16-22 (2003); and Han D, et al., Am. J. Physiol. 278:F628-F634 (2000)).

Increased TGF-• expression in kidney is also associated with kidney transplantation rejection (Shihab F, et al., J. Am. Soc. Nephrol. 6:286-294 (1995); and Shihab F, et al., Kidney Intl. 50:1904-1913 (1996)), various forms of glomerulosclerosis (Yamamoto et al., supra; and YoshiokaK, et al., Lab. Invest. 68:154-163 (1993)), Heyman nephritis (Shankland S, et al., Kidney Intl. 50:116-124 (1996)), remnant kidney (Lee L, et al., J, Clin, invest, 96:953-964 (1995); and Wu L, et al., Kidney Intl. 51:1553-1567 (1997)), ureteral obstruction (Kaneto H, et al., Kidney Intl. 44:313-321 (1993)), kidney diseases caused by radiation and immunosuppressive and nephrotoxic drugs such as cyclosporine, puromycin, cisplatin, and heavy metals (Oikawa T, et al., Kidney Intl. 51:164-172 (1997); Sharma V, e al., Kidney Intl. 49:1297-1303 (1996); Shihab F, et al., Kidney Intl. 49:1141-1151 (1996); Jones C, et al., Am. J. Path. 141:1381-1396 (1992); and Ma L, et al., Kidney Intl. 65:106-115 (2004)), and every animal model of renal injury that has been examined (Noble N & Border W, Sem. Nephrol. 17:455-466 (1997)). Blocking TGF-• activity by its antibodies provided beneficial effects in cyclosporine- and puromycin-induced nephropathies (Ling H, et al., J. Am. Soc. Nephrol. 14:377-388 (2003); Ma L, et al. Kidney Intl. 65:106-115 (2004)).

The mechanism by which TGF-• initiates the development of proteinuria and renal injury, however, is not clear. Identifying downstream respondents of TGF-• in this regard will provide additional and novel targets for the treatment of renal diseases associated with elevations in the expression of TGF-• in the kidney.

### BRIEF SUMMARY

The present invention is summarized as a method for preventing or treating a renal disorder in a human or non-human animal by administering 20-hydroxyeicosatetraenoic acid (20-HETE) or an analog thereof to the human or non-human animal in an amount sufficient to prevent or treat the renal disorder.

The present invention is also summarized as a method for preventing or treating ischemic acute renal failure in a human or non-human animal by administering 20-HETE or an analog thereof to the human or non-human animal in an amount sufficient to prevent or treat ischemic acute renal failure.

The present invention is further summarized as a method for preventing or reducing the severity of damage to an ex vivo preserved kidney upon reperfusion by preserving the kidney ex vivo in a storage solution that contains 20-HETE or an analog thereof in an amount sufficient to prevent or reduce the severity of damage to the kidney upon reperfusion.

### BRIEF DESCRIPTION OF THE DRAWINGS

FIG. 1 shows the expression of TGF-• in the kidney of Sprague Dawley (SD) and Dahl S rats (a genetic model of salt-sensitive hypertension and hypertension-induced renal disease) fed a LS or HS for 7 days. Renal homogenates were isolated from SD (lanes 1-3), Dahl S rats fed a LS diet (lanes 4-7) and Dahl S rats fed a HS diet (8% NaCl) for 7 days (lanes 8-11). Each lane was loaded with a homogenate (30•g protein/lane) isolated from different animals (n=3 to 4 per group). *Indicates a significant difference versus the values seen in Dahl S rats fed a LS diet. HS-7, HS diet for 7 days.

FIG. 2 shows the effect of a HS diet and the role of TGF-on permeability to albumin (P_{alb}) in glomeruli isolated from SD rats and Dahl S rats fed a LS or HS diet for 7 days or from Dahl S rats fed a HS diet that were treated with a TGF-• Ab (1D11-7). The TGF-• Ab used effectively neutralized all three isoforms of TGF-•. Glomeruli were preincubated with vehicle or 10 ng/ml of TGF-• for 15 minutes at 37° C., and P_{alb} was measured. Numbers in parentheses indicate the number of glomeruli and number of rats studied per group. *Indicates a significant difference versus the values seen in Dahl S rats fed a LS diet. Indicates a significant difference from the corresponding control value. HS4, HS for 4 days. HS-7, HS for 7 days.

FIGS. 3a-b show the effect of TGF-• (10 ng/ml) on production of 20-HETE by isolated glomeruli. FIG. 3a shows a representative LC/MS chromatogram showing that TGF-inhibited the formation of a 20-HETE peak with a m/z of 319 that eluted at a retention time of 16 minutes. FIG. 3b summarizes the results from six experiments. Indicates a significant difference from the corresponding control value.

FIG. 4 shows the effect of 20-hydroxyeicosa-5(Z), 14(Z)-dienoic acid (WIT003), a 20-HETE analog, on the changes in P_{alb} produced by TGF-•. Glomeruli were pre-incubated with vehicle or TGF-(10 ng/ml) for 15 minutes at 37° C. and changes in P_{alb} were determined. Glomeruli were pretreated with WIT003 for 15 minutes at 37° C., and the P_{alb} response to TGF-• (10 ng/ml) was redetermined. Numbers in parentheses indicate the number of glomeruli and number of rats studied per group. Indicates a significant difference from the corresponding control value.

FIG. 5 shows a comparison of plasma creatinine concentrations in SD rats following 30-minutes of ischemia and 24 hours of reperfusion of the kidney. Rats were treated with vehicle; N-hydroxy-N′-(4-butyl-2-methylphenol)-formamidine (HET0016, 5 mg/kg), a 20-HETE formation inhibitor; or WIT003 (10 mg/kg) 30 minutes prior to initiation of the ischemia.

FIG. 6 shows a comparison of plasma creatinine concentrations in Dahl S rats (20-HETE deficient strain) and a 2×4 congenic strain of Dahl S rats that overexpress the CYP4A genes that make 20-HETE in the kidney following 20-minutes of ischemia and 24 hours of reperfusion of the kidney.

FIG. 7 shows the plasma concentrations of 20-HETE analogs after a 10 mg/kg s.c. injection of 20-hydroxyeicosa-5(Z), 14(Z)-dienoic acid (5,14-20-HEDE) and N-[20-hydroxyeicosa-5(Z),14(Z)-dienoyl]glycine (5, 14-20-HEDGE) in rats. *P<0.001 vs. the corresponding value in the 5, 14-20-HEDGE treated group.

FIG. 8 shows the effect of 20-HETE analogs on renal dysfunction 24 hours after reperfusion injury. Rats were pretreated with vehicle; HET0016 (5 mg/kg s.c.); 5, 14-20-HEDE (10 mg/kg s.c.); or 5, 14-20-HEDGE (1 mg/kg s.c.). Mean values ±SE are presented. *P<0.05 vs. corresponding value it the vehicle control group. \P<0.05 vs. HET0016.

FIG. 9a-d show renal histology 24 hours after renal ischemia-reperfusion (I/R) injury. FIG. 9a shows hematoxylin and eosin (H&E) stained sections of the renal cortex demonstrating intact (**) and necrotic (*) tubular epithelium following renal Y/R injury. FIG. 9b shows fluorescence microscopy of the same H&E stained field. The necrotic tubules (*) exhibit marked autofluorescence compared to the intact tubules (**). Original magnification, 400×. FIG. 9c shows fluorescence microscopy of representative H&E stained cross-sections of the renal outer medulla are presented. Necrotic tubules (arrowheads) exhibit marked autofluorescence compared to intact tubules (arrows). Original magnification, 100×. FIG. 9d summarizes tubular injury scores 24 hours after renal I/R injury. Pretreatment of rats with 5, 14-20-HEDE or 5,14-20-HEDGE resulted in less severe renal injury compared to vehicle controls or rats treated with HET0016. Mean values ±SE are presented. *P<0.05 vs. vehicle-treated group. \P<0.05 vs. HET0016.

FIGS. 10a-c show the effect of 5, 14-20-HEDGE on apoptosis 24 hours after renal I/R injury, in which apoptosis of renal tubular epithelial cells was assessed by TUNEL staining. FIG. 10a shows sections from vehicle-treated rats. FIG. 10b shows sections from rats treated with 5, 14-20-HEDGE (1 mg/kg, s.c.). FIG. 10c shows the number of apoptotic nuclei was significantly less in 5, 14-20-HEDGE-treated rats when compared to vehicle-treated controls. Mean values ±SE are presented. *P<0.05 vs. vehicle-treated group.

FIGS. 11a-c show the effect of 5, 14-20-HEDGE on renal hemodynamics after renal UR injury. FIG. 11a shows the effect of 5, 14-20-HEDGE (1 mg/kg s.c.) on cortical blood flow (CBF) in rats during and following renal I/R injury. FIG. 11b shows the effect of 5,14-20-HEDGE (1 mg/kg s.c.) on medullary blood flow (MBF) in rats during and following renal I/R injury. FIG. 11 c shows the effect of 5, 14-20-HEDGE (1 mg/kg s.c.) on mean arterial pressure (MAP) in rats during and following renal T/R injury. Values are represented as mean ±SE. *P<0.05 vs. the corresponding value in the vehicle-treated group. \P<0.001 vs. the corresponding value in the vehicle-treated group.

While the present invention is susceptible to various modifications and alternative forms, exemplary embodiments thereof are shown by way of example in the drawings and are herein described in detail. It should be understood, however, that the description of preferred embodiments is not intended to limit the invention to the particular forms disclosed, but on the contrary, the intention is to cover all modifications, equivalents and alternatives falling within the spirit and scope of the invention as defined by the appended claims.

### DETAILED DESCRIPTION OF PREFERRED EMBODIMENTS

The present invention relates to the inventors' observation that upregulation of renal TGF-• increased permeability of the glomerular filtration barrier to albumin and other macromolecules through inhibition of glomerular production of 20-BETE. Increased glomerular permeability to albumin led to proteinuria and further to other glomerular injuries (e.g., glomerulosclerosis and renal interstitial fibrosis). The present invention therefore provides new tools for preventing and/or treating TGF•-related renal disorders, as well as physical and pathological manifestations thereof.

In one aspect, the present invention relates to a method for preventing or treating a TGF•-related renal disorder in a human or non-human animal. The method involves administering 20-HETE or a 20-HETE analog to the human or non-human animal in an amount sufficient to prevent or treat the renal disorder. By “TGF•-related renal disorder,” we mean a renal disease and physical and pathological manifestations thereof in which TGF-• expression is upregulated. Examples of such disorders include, but are not limited to, proteinuria, nephropathies induced by diabetes and hypertension (e.g., salt sensitive hypertension), kidney transplantation rejection, Heyman nephritis, remnant kidney nephropathy, ureteral obstruction nephropathy and kidney diseases caused by radiation and immunosuppressive and nephrotoxic drugs, such as acetomenophen, adriamycin, cyclosporine, gentamicin, puromycin, cisplatin and heavy metals. Also included with this definition is renal injury that may result following administration of contrast medias for angiograms and other imaging studies, as well as Escherichia coli.

In one embodiment, the method of the present invention can be employed to prevent or treat proteinuria or a proteinuria-related renal disorder. By “proteinuria-related renal disorder,” we mean a renal disease in which proteinuria is detected. In another embodiment, the method of the present invention can be employed to prevent or treat diabetes- or hypertension-induced nephropathy. In yet another embodiment, the method of the present invention can be employed to prevent or treat ischemic acute renal failure and other forms of ischemic renal injury such as that arising following administration of contrast media (i.e., contrast media nephropathy) or exposure to nephrotoxic drugs and other substances.

Examples of 20-HETE analogs that can be used in the present invention include, but are not limited to those disclosed in U.S. Pat. No. 6,395,781; Yu M, et al., Eur. J. Pharmacol. 486:297-306 (2004); Yu M, et al., Bioorg. Med. Chem. 11:2803-2821 (2003); and Alonso-Galicia M, et al., Am. J. Physiol. 277:F790-796 (1999), each of which is incorporated herein by reference as if set forth in its entirety. For example, 20-HETE analogs defined by the following formula as provided in U.S. Pat. No. 6,395,781 can be used in the present invention:

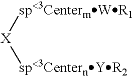

wherein R₁ is selected from the group consisting of carboxylic acid, phenol, amide, amine (including any amino acid), imide, sulfonamide, sulfonamide, active methylene, 1,3-dicarbonyl, alcohol, thiol, tetrazole and other heteroaryl groups;

wherein R₂ is selected from the group consisting of carboxylic acid, phenol, amide, amine (including any amino acid), imide, sulfonamide, sulfonamide, active methylene, 1,3-dicarbonyl, alcohol, thiol, tetrazole and other heteroaryl groups;

wherein W is a carbon chain (C₁ through C₂₅) and may be linear, cyclic or branched and may comprise heteroatoms;

wherein Y is a carbon chain (C₁ through C₂₅) and may be linear, cyclic or branched and may comprise heteroatoms;

wherein sp^{<3} Center is selected from the group consisting of vinyl, aryl, heteroaryl, cyclopropyl and acetylenic moieties;

wherein X is an alkyl chain that may be linear, branched, cyclic or polycyclic and may comprise heteroatoms;

wherein m is 0, 1, 2, 3, 4 or 5; and wherein n is 0, 1, 2, 3, 4 or 5.

Preferably, a 20-HETE analog defined by the above formula has a carboxyl or other ionizable group at either R₁ or R₂ and contains a double bond or other functional group at a distance equal to 14-15 carbons from the ionizable group (U.S. Pat. No. 6,395,781). More preferably, the 20-HETE analog contains a length of 20-21 carbons, has a carboxyl or other ionizable group at either R₁ or R₂, contains a double bond or other functional group at a distance equal to 14-15 carbons from the ionizable group, and contains a hydroxyl group on the 20 or 21 carbon at either R₁ or R₂ (U.S. Pat. No. 6,395,781).

In one form, the present invention contemplates the use of the following 20-HETE analog: 20-hydroxyeicosa-5(Z),14(Z)-dienoic acid (5, 14-20-HEDE), the structure of which is shown below:

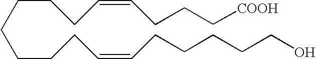

In another form, the present invention contemplates the use of modifications of the carboxyl group on C1 to make it more soluble such as the following 20-HETE analog: N-[20-hydroxyeicosa-5(Z),14(Z)-dienoyl]glycine (5, 14-20-HEDGE), the structure of which is as follows:

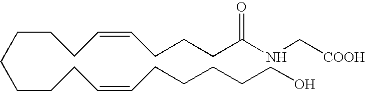

The inventors contemplate that any 20-HETE analog can be used with the methods described herein, including C₁ carboxy derivatives of 20-HETE and methyl sulfonyl derivatives of 5, 14-20-HEDE.

The present invention is not limited by a specific route of administration. Suitable routes of administration for 20-HETE or a 20-HETE analog include, but are not limited to, oral administration, intravenous administration, subcutaneous administration, intramuscular administration, intrarenal artery administration, as well as direct delivery into renal interstitium or through sustained release into the blood via drug eluting vascular stents, coupling the drugs into liposomes or dextrans or proteins that are filtered and accumulated by renal tubular epithelial cells or impregnating the compounds on microbeads or particles that become trapped in the renal microvasculature and slowly dissolve.

Optimal dosages of 20-HETE or a particular 20-HETE analog for preventing or treating a particular renal disorder via a particular route of administration can be readily determined by one of ordinary skill in the art, but preferably can be in the range from 0.1 to 30 mg/kg.

The invention will be more fully understood upon consideration of the following non-limiting examples.

### EXAMPLES

### Example 1

### 20-HETE Analog Opposes TGF•-Induced Glomerular Injury

This example shows that TGF-alters the glomerular permeability by inhibiting the glomerular production of 20-HETE. Renal expression of TGF-• doubled in Dahl S rats fed a high salt diet for 7 days, which was associated with a marked rise in permeability to albumin (P_{alb}) from 0.19±0.04 to 0.75±0.01, along with changes in the ultrastructure of the glomerular filtration barrier. Chronic treatment of Dahl S rats with a TGF-• neutralizing antibody prevented the increase in P_{alb} and preserved the structure of glomerular capillaries proving that hypertension-induced renal disease is dependent on increased formation and action of TGF-•. It had no effect on the rise in blood pressure produced by the high-salt diet. Preincubation of glomeruli isolated from SD rats with TGF-•1 (10 ng/ml) for 15 minutes increased P_{alb} from 0.01±0.01 to 0.60±0.02. This was associated with inhibition of the glomerular production of 20-HETE from 221±11 to 3.4±0.5·g/30 minutes/mg protein. Pretreatment of SD glomeruli with WIT003 reduced baseline P_{alb} and opposed the effects of TGF-• to increase P_{alb}.

Materials and Methods.

Dahl salt-sensitive rat model: Dahl S rats exhibit many traits associated with salt-sensitive hypertension in humans (Campese V, Hypertension 78:531-550 (1994); and Grimm C, et al., Hypertension 15:803-809 (1990)). They are salt-sensitive (Iwai, J, Hypertension 9:118-120 (1987); and Rapp J, Hypertension 4:753-763 (1982)), insulin-resistant (Reft G, et al., Hypertension 18:630-635 (1991)) and hyperlipidemic (Raji L, et al., Kidney IntL. 41:801-806 (1984); and O'Donnell M, et al., Hypertension 20:651-658 (1992)), and they rapidly develop proteinuria and glomeruloscierosis when challenged with a high salt (HS) diet (O'Donnell et al., supra; Roman R, et al., Hypertension 12:177-183 (1988); Roman R, et al., Hypertension 21:985-988 (1988); Roman R, et al., Am. J. Hypertens. 10:63 S-67S (1997); and Tolins J, et al., Hypertension 16:452-461 (1990)). Glomerular lesions that develop resemble those seen in patients with hypertension- and diabetes-induced nephropathy (McClellan W, et al., Am. J. Kidney Dis. 12:285-290 (1987); Ronstand G, et al., N. Engl. J. Med. 306:1276-1279 (1982); and Tierney W, et al, Am. J. Kidney. Dis. 13:485-493 (1989)). Moreover, renal expression of TGF-• is elevated in Dahl S rats fed a high salt (HS) diet and chronic treatment of Dahl S rats with a TGF-• neutralizing antibody (Ab) for three weeks reduces proteinuria and the degree of glomerulosclerosis and fibrosis (Dahly et al., supra).

General Methods: Experiments were performed on 7-week-old SD rats (Taconic Labs; Hudson, N.Y.) fed a normal-salt diet containing 1% NaCl (#5010; Purina Mills; St. Louis, Mo.) and Dahl salt-sensitive/John Rapp rats obtained from a colony maintained at the Medical College of Wisconsin (Milwaukee, Wis.). Rats were fed a purified diet (AlN76) purchased from Dytes, Inc. (Bethlehem, Pa.) that contained either 0.4% (low salt, LS) or 8.0% NaCl (high salt, HS). To assess the role of TGF-• in altering proteinuria and P_{alb} during hypertension development, a group of the Dahl S rats fed a HS diet were treated with an intraperitoneal injection of a murine anti-TGF-monoclonal Ab (0.5 mg/kg; 1D11; Genzyme Corp.; Cambridge, Mass.) or a control murine monoclonal Ab (13C4; antiverotoxin) every other day (Dasch J, et al., J. Immunol. 10:2109-2119 (1989)). At the end of the treatment period, rats were placed overnight in metabolic cages for measurement of protein and albumin excretion (Dahly et al., supra). They were then anesthetized with halothane, and the kidneys were collected for measurement of the expression of TGF-protein levels by Western blot (Hoagland K, el al., Hypertension 43:860-865 (2004)) and for glomerular isolation for the measurement of P_{alb} and the production of 20-HETE. Catheters connected to radiotelemetry transmitters (Data Science Inc.; St. Paul, Minn.) were implanted into the femoral artery of 10 additional control and 10 D11-treated Dahl S rats to determine the effects of anti-TGF-• therapy on the development of hypertension. Mean arterial pressure (MAP) was measured for 3 hours per day, between 9 AM and 12 PM, during a control period when rats were fed a LS diet and after they were fed a HS diet for 7 days.

Measurement of Albumin Permeability (P_{alb}): Glomeruli were isolated using the sieving method as previously described in a medium containing 5 g/dl of bovine serum albumin (BSA). See, Sharma R, et al, Kidney Intl. 58:131-136 (2000); and Savin V, et al. J. Am. Soc. Nephrol. 3:1260-1269 (1992). In each experimental condition, P_{alb} was determined from the change in glomerular volume (•V) after exchange of the bath with medium containing 1 g/dl albumin. P_{alb} was calculated as 1−(•Vₑₓₚₑᵣᵢₘₑₙₜₐₗ/•V_{control}) where glomeruli from SD rats fed a normal-salt diet were used to provide the control value for each experiment. To verify that lack of •Vs in Dahl S rats were related to changes in P_{alb} rather than to changes in mechanical properties of glomeruli, additional studies were performed in which the glomeruli were exposed to a 5% solution of high molecular weight dextran. A change in the size of Dahl S glomeruli under these conditions indicates that the lack of response to 1% albumin was attributable to an increase in P_{alb} (Savin et al., supra).

In other experiments, we examined the interaction of TGF-• and 20-HETE on P_{alb} in glomeruli isolated from SD rats and Dahl S rats fed either a LS diet or a HS diet for 4 days. Glomeruli were preincubated with vehicle or TGF-•1 (10 ng/ml) for 15 minutes at 37° C. and changes in P_{alb} were determined. Glomeruli were also pretreated with 5, 14-20-HEDE (WIT003) (1 μmol/L; Taisho Pharmaceutical; Tokyo, Japan) (Alonso-Galicia et al., supra; and Yu et al., supra), for 15 minutes at 37° C. and the P_{alb} response to TGF-• 1 (10 ng/ml) was redetermined. A minimum of 5 glomeruli from each rat were studied, and the experiments were performed using •5 rats per treatment group.

Electron microscopy: Kidneys from Dahl S rats fed a LS diet and Dahl S rats fed a HS diet for 1 week and treated with 1D11 or vehicle were collected and fixed in a 4% glutaldehyde solution. Thin epon sections were prepared, stained with uranyl acetate and lead citrate, and examined at 16,000× using a transmission electron microscope (Hitachi H600).

Western blots: Homogenates were prepared from the kidneys of control SD rats and Dahl S rats fed a LS or HS diet for 7 days. Aliquots of the homogenates (30 μg protein) were separated on a 12.5% sodium dodecyl sulfate gel, transferred to a nitrocellulose membrane incubated with a primary TGF-•1 Ab (SC: 146; Santa Cruz Biotechnology; Santa Cruz, Calif.), followed by a secondary Ab (SC:2004; Santa Cruz Biotechnology) and developed using enhanced chemiluminescence as previously described. See, Hoagland K, et al., Hypertension 43:860-865 (2004). Membranes were post-stained with Commassie blue to normalize results for potential differences in sample loading.

Liquid Chromatography/Mass Spectroscopy measurement of glomerular 20-HETE production: Glomeruli (approximately 20 μg protein) were incubated in a 0.1 mol/L KPO₄ buffer containing 1 mmol/L NADPH for 30 minutes at 37° C. in the presence and absence of TGF-•1 (10 ng/ml). Incubations were stopped by acidification with formic acid, homogenized, and the homogenate extracted with chloroform:methanol (2:1) after addition of 10 ng of internal standard, 14,15-epoxyeicosa-5(Z)-enoic-methyl sulfonylimide (EEZE). Samples were reconstituted in 50% acetonitrile, cleaned up using on online, reverse-phase high performance liquid chromatography (HPLC) trapping column, and then the HETEs and epoxyeicosatrienoic acids (EETs) in the samples were separated using an isocratic step gradient on an 18C—RP 2×250 mm microbore HPLC (BetaBasic18 150×21 3•m; Thermo Hypersil-Keystone; Bellefonte, Pa.) using a mobile phase consisting of acetonitrile:water:acetic acid (57:43:0.1) for 20 minutes to resolve the HETEs followed by acetonitrile:water:acetic acid (63:37:0.1) for 15 minutes to resolve the EETs. Samples were ionized using negative ion electrospray and the peaks eluting with a mass/charge ratio (m/z) of 319 (HETEs and EETs) or 323 (internal standard) were isolated and monitored in the selective ion mass spectroscopy (MS) mode using an Agilent LSD ion trap mass spectrometer (Agilent Technologies 1100; Santa Clara Calif.). The ratio of ion abundances in the peaks of interest (HETEs and EETs, m/z 319) versus that corresponding to the closely eluting internal standard (EEZE, m/z 323) were determined and compared with a standard curve generated over a range from 0.1 ng to 2 ng of 20-HETE and EETs with each batch of samples.

Statistics: Mean values ±1 SE are presented. Significance of differences between mean values was determined using an ANOVA followed by the Student-Newman-Keuls post hoc test. A P<0.05 was considered significant.

Results.

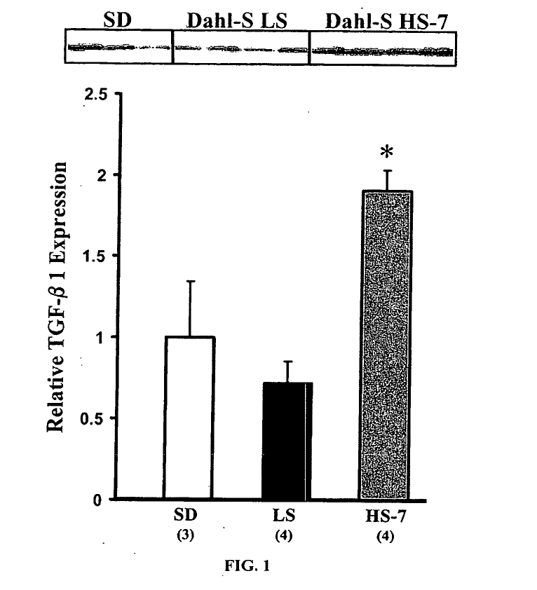

Effects of high salt diet on the renal expression of TGF-•1: The results are presented in FIG. 1. The expression of TGF-•1 in the kidney more than doubled in Dahl S rats fed a HS diet for 1 week compared with the levels seen in Dahl S rats fed a LS diet.

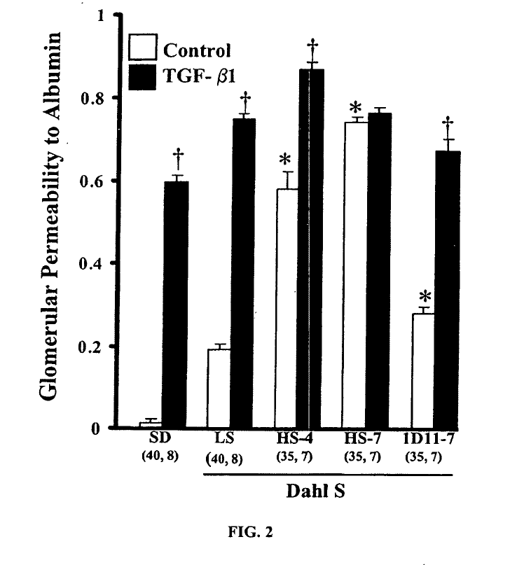

Effects of high salt diet on P_{alb}: A comparison of P_{alb} in SD and Dahl S rats fed a LS and HS diet at various times for up to a week are presented in FIG. 2. Baseline P_{alb} was significantly higher in Dahl S rats maintained on a LS diet than in control SD rats. P_{alb} increased in Dahl S rats fed a HS diet after only 4 days, and it reached a peak after 7 days. The increase in P_{alb} in Dahl S rats fed a HS diet for one week was associated with a significant rise in blood pressure from 121±2 to 136±3 mm Hg (n=10) and a marked increase in the excretion of protein from 47±8 mg/day to 217±31 mg/day (n=14). Similarly, albumin excretion rose from 27±9 mg/day to 129±26 mg/day, after Dahl S rats were fed a HS diet for 7 days.

Role of TGF-• in alterting P_{alb} in Dahl S rats: A comparison of the effects of exogenous administration of TGF-•1 (10 ng/ml) on P_{alb} in glomeruli isolated from SD and Dahl S rats is also summarized in FIG. 2. TGF-•1 increased P_{alb} from 0.01±0.01 to 0.56±0.02 in glomeruli isolated from SD rats and from 0.19±0.01 to 0.75±0.01 in glomeruli isolated from Dahl S rats fed a LS diet. TGF-•1 also increased in P_{alb} in Dahl S rats fed a HS diet for 4 days, but it had no effect on P_{alb} in Dahl S rats fed a HS diet for 7 days, because the baseline P_{alb} in these rats was already near maximal.

Interestingly, chronic treatment of Dahl S rats fed a HS diet with a TGF-• neutralizing Ab prevented the increase in baseline P_{alb}. Administration of TGF-•1 to these glomeruli still increased P_{alb}, similar to that seen in glomeruli isolated from control SD rats and Dahl S rats fed a LS diet. TGF-•1 Ab therapy had no effect on the rise in blood pressure. Blood pressure rose from 123±4 to 136±3 mm Hg (n=10) in Dahl S rats fed a HS diet that were treated with 1D11 for 7 days.

Electron microscopy: Electron micrographs of the ultrastructure of glomerular capillaries in Dahl S rats fed a LS or HS diet, and in those treated with the TGF-• Ab for 1 week, were obtained. The Dahl S rats fed the LS diet exhibited a normal appearance of the glomerular ultrafiltration barrier. In Dahl S rats fed the HS diet for 7 days, there was a retraction and fusion of the foot processes of podocytes and exposure of portions of the basement membrane. There was also swelling of the endothelial cells lining the glomerular capillaries, which changed their shape from a flattened to a more cubodial endothelium. These changes in the ultrastructure of glomerular filtration barrier in Dahl S rats fed a HS diet were prevented by administration of the TGF-• Ab.

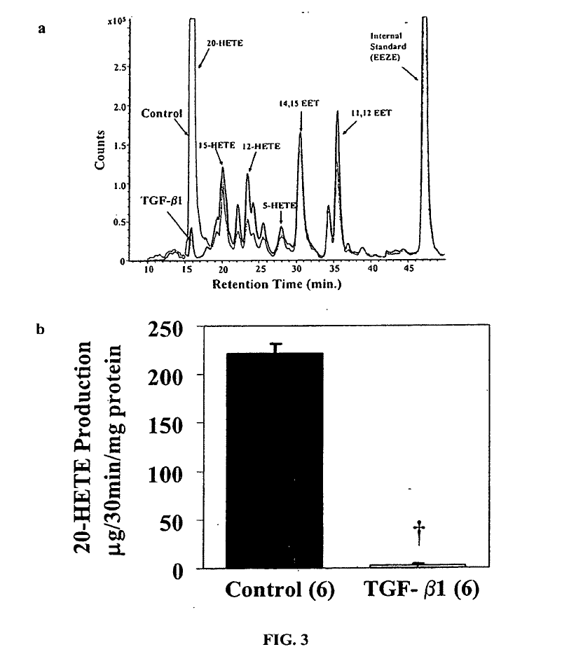

Effect of TGF-• on the glomerular production of 20-HETE: The effects of TGF-• on the production and metabolism of arachidonic acid (AA) by isolated glorneruli are presented in FIG. 3. Glomeruli incubated with AA produced a number of large peaks with an m/z of 319 that co-elutes with 20-HETE; 15-HETE; 12-HETE; 5-HETE; 14,15-EET; 11,12-EET; 8,9-EET and 5,6-EET standards (FIG. 3a). We further verified that the largest peak that elutes at 16 minutes after fragmentation produces an MS/MS spectrum with prominent secondary ions at m/z of 301, 273, 257, and 245, identical to that seen with a 20-HETE standard. Pretreatment of glomeruli with TGF-•1 selectively reduced the formation of 20-HETE by 97% (FIG. 3b) without affecting the formation of 15-, 12- or 5-HETE or EETs (FIG. 3a).

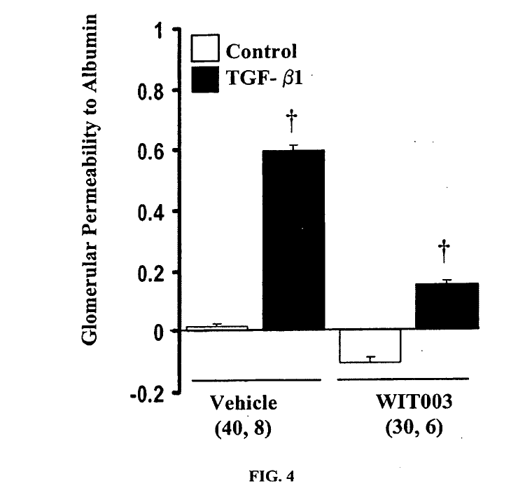

Effects of a 20-HETE analog on P_{alb}: The effect of addition of a 20-HETE analog on the changes in P_{alb} produced by TGF-•1 is summarized in FIG. 4. Pretreatment of glomeruli with a 20-HETE analog reduced baseline P_{alb} and greatly attenuated the increase in P_{alb} produced by TGF-•1. Similar results were obtained with Dahl S rats maintained on the LS diet or fed the HS diet for 4 days. For example, TGF-•1 increased P_{alb} from 0.58±0.04 (n=25 glomeruli; 5 rats) to 0.87±0.02 (n=25; 5 rats) in glomeruli isolated from Dahl S rats fed the HS diet for 4 days. After pretreatment of glomeruli with the 20-HETE analog, TGF-•1 P_{alb} only increased from 0.25±0.01 (n=25; 5 rats) to 0.40±0.01 (n=25; 5 rats).

### Example 2

### Protection of Kidney from Ischemic Injury by 20-HETE and 20-HETE Analogs

This example shows the effect of the 20-HETE analog, 5, 14-20-HEDE (WIT003), on protecting the kidney from renal I/R injury. 5, 14-20-HEDE significantly reduced the degree of renal I/R injury reflected by preventing a rise in creatinine concentration.

Materials and Methods.

Renal Ischemia-Reperfusion Injury Model: Experiments were performed in male SD rats anesthetized with pentobarbital (50 mg/kg). The kidneys were exposed via a midline incision and the renal arteries isolated. Adjustable vascular occluders were placed on both the right and left renal arteries to completely occlude blood flow to the kidneys for 30 minutes. After the period of complete renal ischemia, the clamps were removed and the kidneys were reperfused. The surgical incisions were closed with 2-0 silk suture and the animals were allowed to fully recover from anesthesia. Twenty four hours later, the rats were reanesthetized with pentobarbital, and a sample of blood collected from the aorta for measurement of plasma creatinine concentration using an autoanalyzer. The kidneys were collected, fixed in 10% formalin solution, and paraffin sections prepared and stained with H&E to evaluate the degree of tubular necrosis and injury.

Three groups of rats were studied. Group 1 rats were treated with vehicle and served as the control animals. Group 2 rats were treated with HET0016 (5 mg/kg, s.c.) 30 minutes prior to renal ischemia. Group 3 rats were given 5, 14-20-HEDE (WIT003) (10 mg/kg, s.c.) by injection 30 minutes prior to renal ischemia.

Results.

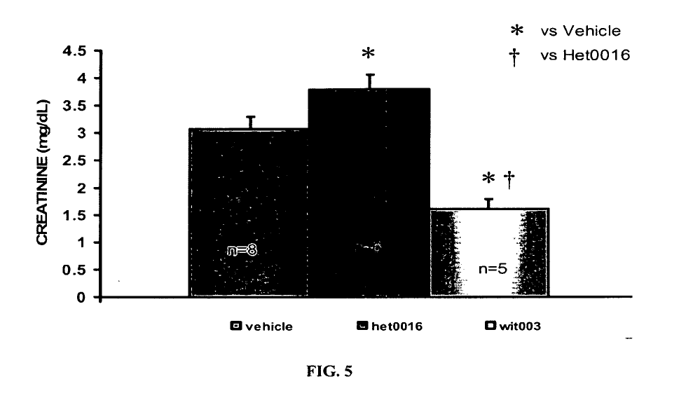

FIG. 5 shows the results of the in vivo experiments in which the effects of HET0016 and 5, 14-20-HEDE (W1T003) on the degree of renal injury following ischemia and reperfusion of the kidney were examined. Plasma creatinine levels rose from 0.5 to approximately 3.0 mg/dl 24 hours after the kidney of SD rats was subjected to 30 minutes of complete ischemia followed by 24 hours of reperfusion. The degree of injury, reflected by the rise in plasma creatinine concentration, was significantly greater in rats treated with HET016 (5 mg/kg, s.c.), given 30 minutes prior to ischemia. Administration of 5, 14-20-HEDE (WIT003) (10 mg/kg, s.c.), 30 minutes prior to reperfusion, significantly reduced the degree of renal I/R injury reflected by the rise in creatinine concentration. The rise in plasma creatinine concentration following ischemia reperfusion in the control animals is associated with severe necrosis of the S3 segment of the proximal tubule. The degree of histological damage to this segment of the renal tubules was reduced in rats treated with the 20-HETE analog (data not shown).

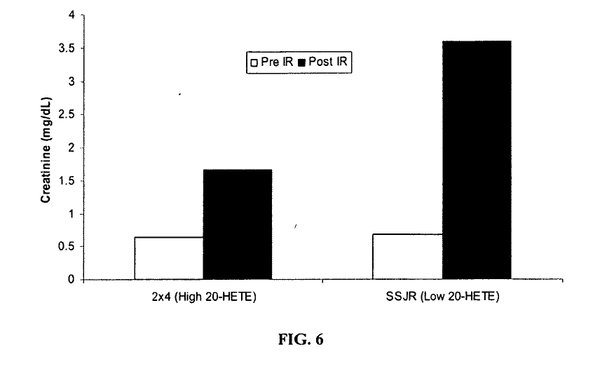

In other experiments, we compared the degree of renal injury seen in Dahl S rats (20-HETE deficient strain) subjected to 20 minutes of ischemia reperfusion with that seen in a congenic strain of Dahl S rat called 2×4 in which we introduced the CYP4A gene from Lewis rats that encodes for the enzyme that produces 20-HETE in the kidney. Transfer of this gene upregulates the expression of CYP4A protein in the kidney and the production of 20-HETE in the kidney. As can be seen in FIG. 6, transfer of the CYP4A gene from the Lewis rat into the Dahl S genetic background also significantly reduced the degree of renal damage as reflected by the lesser rise in plasma creatinine concentration 24 hours after ischemia and reperfusion. This data is therefore consistent with the results obtained in the SD rats that upregulation of the endogenous formation of 20-HETE or administration of a 20-HETE analog protects the kidney against ischemic renal injury, while inhibition of the renal formation of 20-HETE exacerbates the degree of injury.

### Example 3

### Protection of Kidney from Ischemic Injury by Additional 20-HETE Analogs

This example shows that the 20-HETE analogs, 5, 14-20-HEDE and 5,14-20-HEDGE, reduced renal injury and that the reduced renal injury was associated with preserved renal medullary blood flow following renal I/R injury. Administration of HET0016, a selective inhibitor of the renal formation of 20-HETE, exacerbated renal injury and plasma creatinine rose to 3.8±0.3 mg/dl. Conversely, administration of 5,14-20-HEDE or 5,14-20-HEDGE attenuated renal injury, and plasma creatinine only rose to 1.6±0.3 mg/dl and 0.5±0.02 mg/dl, respectively. In control rats, MBF measured by laser-Doppler flowmetry decreased to 50% of baseline 1 hour after reperfusion. 5, 14-20-HEDGE completely prevented the post-ischemic fall in MBF without altering MAP or CBF. 20-HETE and its analogs therefore appear to protect the kidney from renal I/R injury by preventing a post-ischemic fall in MBF and provide a rationale for the development of 20-HETE analogs as novel preventative or therapeutic agents in renal I/R injury.

Materials and Methods.

General Methods: Experiments were performed on male SD rats weighing ˜300 g (Taconic Farms; Germantown, N.Y.). The SD rats were housed in an Animal Care Facility at the Medical College of Wisconsin, which is approved by the American Association for the Accreditation of Laboratory Animal Care. All protocols were approved by the Institutional Animal Care and Use Committee of the Medical College of Wisconsin.

Renal I/R Injury Model: Rats were anesthetized with ketamine (50 mg/kg, i.m.) and sodium pentobarbital (50 mg/kg, i.p) and were placed on a heated surgical table to maintain body temperature at 37° C. A midline abdominal incision was made to expose the kidneys, and the renal arteries and veins were bilaterally occluded for 30 minutes using microvascular clamps. The clamps were then removed, the abdominal incision was closed, and the rats were allowed to recover for 24 hours. Rats were then euthanized with sodium pentobarbital (100 mg/kg, i.p.), and blood was collected from the aorta for measurement of plasma creatinine concentration using the Jaffe reaction. Both kidneys were collected for histological analysis.

In addition, the method was repeated on four groups of rats. However, rats were pretreated with a s.c. injection of vehicle or one of three treatments 30 minutes prior to induction of renal ischemia. Group 1 served as the control group and received 1 ml/kg of a vehicle (11% sulfobutyl-•-cyclodextrin in 165 mM mannitol solution, 0.1 ml/kg s.c.); Group 2 received HET0016 (5 mg/kg, s.c.); Group 3 received 5, 14-20-HEDE (10 mg/kg; s.c.); and Group 4 received 5, 14-20-HEDGE (1 mg/kg; s.c.).

Measurement of plasma levels of the 20-HETE analogs: A chronic polyvinyl catheter was implanted into the jugular vein and exteriorized at the back of the neck of SD rats several days prior to an experiment. After a three-day recovery period, the rats (n=4 in each group) received a s.c. injection of either 5, 14-20-HEDE (10 mg/kg) or 5, 14-20-HEDGE (10 mg/kg), and blood samples were collected 1, 4, 8 and 24 hours after administration. Plasma levels of 5, 14-20-HEDE and 5, 14-20-HEDGE were measured by liquid chromatography-mass spectrometry (LC/MS) using an Applied Biosystems API 3000 LC/MS/MS (Foster City, Calif.).

Analysis of the degree of tubular injury: The kidneys of rats subjected to renal I/R injury were fixed in 10% formalin and paraffin sections (3•m) were prepared and stained with hematoxylin and eosin. The sections were examined at low power using a 2× objective of an Olympus BHT-2 (Olympus; Center Valley, Pa.) epifluorescent microscope equipped with a 540-nm excitation filter and a 590-nm emission filter. For each specimen, five randomly chosen outer medullary fields were photographed at 100× magnification using a digital color camera and following appropriate thresholding. The percentage of the area containing fluorescent necrotic tubular epithelium or cast material was quantified using Image-Pro Plus image analysis software (version 6.2; Media Cybernetics, Inc.; Bethesda, Md.). TUNEL labeling was also performed to assess the degree of apoptosis following renal LIR injury using an ApopTag Plus In Situ Apoptosis Fluorescein Detection Kit (Chemicon International; Temecula, Calif.) following the manufacturer's protocol. Apoptotic nuclei were visualized at approximately 100× by fluorescence microscopy and quantified using Metamorphi Image Analysis Software (Molecular Devices Corp.; Dowington, Pa.). A total of five fields were analyzed per section, with one section analyzed per animal. Data were expressed as the percentage of TUNEL positive nuclei per total number of DAPI stained cells.

Assessment of renal hemodynamics: Additional experiments were performed to determine if the renoprotective effect of 5, 14-20-HEDGE on renal I/R injury was associated with changes in renal cortical and medullary hemodynamics. Rats were anesthetized with ketamine (50 mg/kg, i.m.) and Inactin (100 mg/kg, i.p.; Sigma; St. Louis, Mo.). Catheters were placed in the femoral artery for continuous measurement of arterial pressure and in the femoral vein for i.v. infusions. The left kidney was then exposed and placed in a kidney holder and a single-mode optical fiber was implanted 4 mm into the kidney for measurement of MBF by laser-Doppler flowmetry (LDF) as previously described. See, Zou A, et al., J. Hypertens. 13:557-566 (1995). CBF was measured by a second laser-Doppler probe held in static position by a micromanipulator 1 mm above the renal cortex. After surgery and a 30-minute stabilization period, MAP, CBF and MBF were continuously recorded using WinDaq data acquisition software (DATAQ Instruments Inc.; Akron, Ohio) during a 5-minute control period. Baseline MAP, MBF and CBF were determined and then 5, 14-20-HEDGE (1 mg/kg s.c.) or vehicle (1 ml/kg s.c.) were administered. Thirty minutes later, the blood supply to the left kidney was occluded for 45 minutes. MAP, MBF and CBF were recorded during ischemia and for 3 hours following reperfusion.

Statistical Analysis: Mean values ±SE are presented. The significance of differences in mean values between groups was analyzed using a one-way ANOVA or repeated measures ANOVA with Dunns post-hoc test. A P value <0.05 was considered to be statistically significant.

Results.

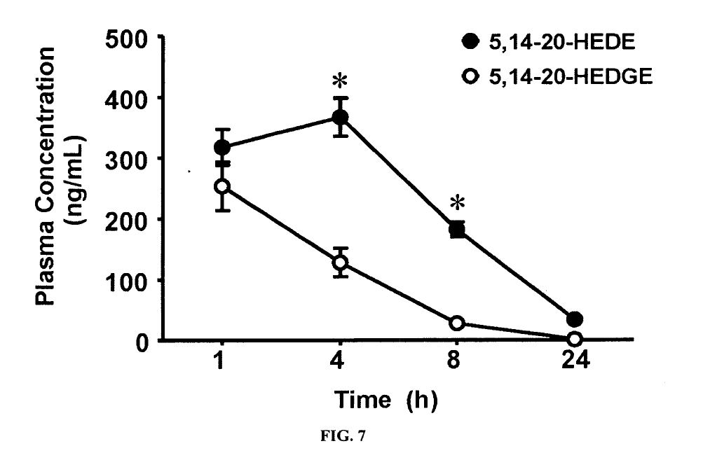

Plasma concentrations of 20-HETE analogues following subcutaneous injection: Plasma concentrations of 5,14-20-HEDE and 5,14-20-HEDGE at 1, 4, 8 and 24 hours after injection are presented in FIG. 7. Peak blood levels were similar after administration of 10 mg/kg of either compound and averaged 300 ng/ml or approximately 1 μM. The half-lives of the two analogs were also similar and averaged approximately four hours.

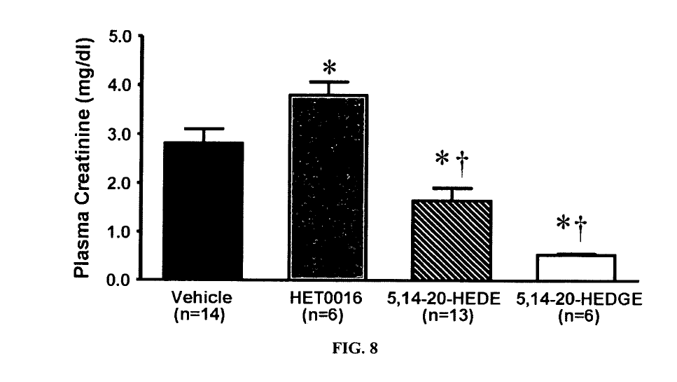

Effect of 20-HETE on renal I/R injury: In vehicle-treated control rats, plasma creatinine increased from 0.5±0.05 mg/dl to 2.8±0.3 mg/dl 24 hours after ischemia. Blockade of the formation of 20-HETE by HET0016 exacerbated renal dysfunction as evidenced by a significant increase in plasma creatinine levels. Pretreatment of the rats with 5, 14-20-HEDE (10 mg/kg) reduced plasma creatinine by approximately 50%. 5, 14-20-HEDGE was more efficacious than 5, 14-20-HEDE in that a 10-fold lower dose was able to nearly completely prevent the rise in plasma creatinine 24 hours following renal I/R injury (FIG. 8).

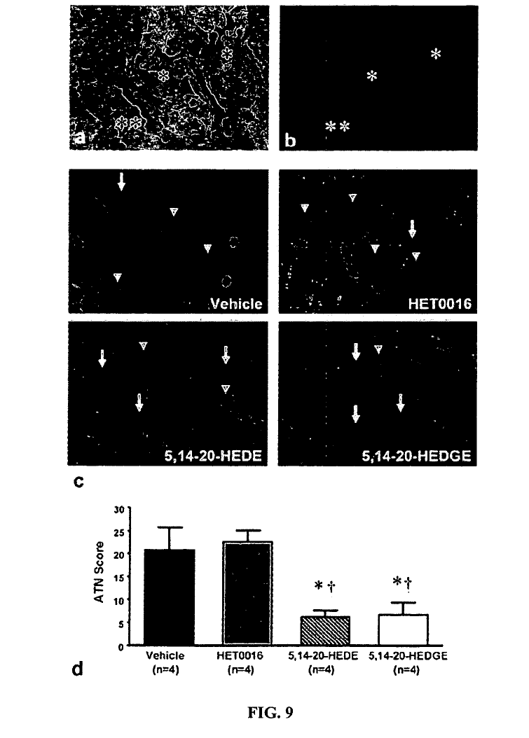

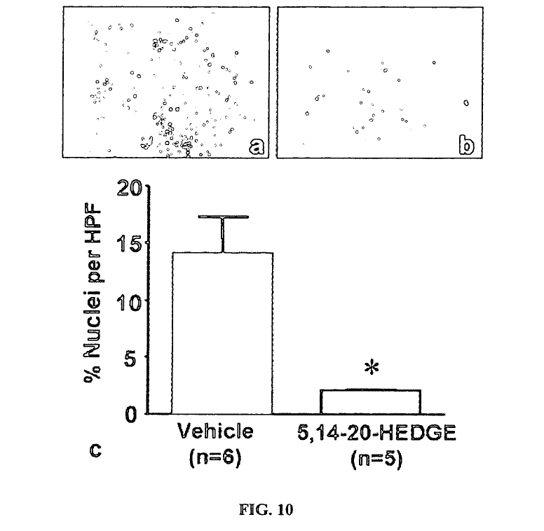

Effects of 20-HETE analogues on renal tubular injury and apoptosis following I/R: The histological appearance of tubular injury at the corticomedullary junction is presented in FIG. 9. Fluorescence microscopy of the H&E stained sections revealed distinct autofluorescence of necrotic tubules and tubular casts (FIGS. 9a and 9b). Diffuse tubular cell denudation, tubular cell necrosis, intratubular debris and tubular casts were present in vehicle- and HET016-treated animals (FIG. 9c). Pretreatment with a 5, 14-20-HEDE or 5, 14-20-HEDGE resulted in less severe renal injury with only focal tubular necrosis or exfoliation of tubular cells (FIG. 9c). The degree of renal injury was further quantified by morphometric analysis of autofluorescence in H&E stained sections. Pretreatment of rats with 5, 14-20-HEDE or 5,14-20-HEDGE resulted in a marked reduction in the area occupied by necrotic tubular epithelium or cast material compared to vehicle or RET0016-treated animals (FIG. 9d). Furthermore, pretreatment with a 20-HETE analogue markedly reduced the number of apoptotic cells identified by TUNEL staining compared to vehicle treated controls (FIG. 10).

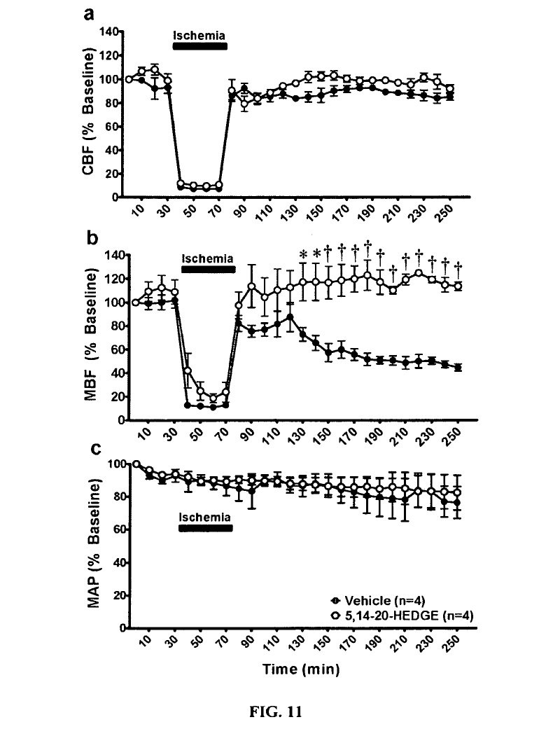

Effect of 5,14-20-HEDGE on renal hemodynamics: Baseline CBF and MBF were similar in rats that received vehicle or 5,14-20-HEDGE. 5,14-20-HEDGE did not have a significant effect on CBF or MBF prior to the induction of renal ischemia. As expected, CBF and MBF fell by greater than 90% in both groups during the ischemic period and recovered after reperfusion. In vehicle-treated rats, MBF slowly decreased to approximately 50% of baseline by 60 minutes following reperfusion and remained at that level until termination of the experiment. In contrast, pretreatment of the rats with 5, 14-20-HEDGE prevented the post-ischemic fall in MBF (FIG. 11b). 5, 14-20-HEDGE had no effect on post-ischemic CBF (FIG. 11a) or MAP (FIG. 11c) when compared to vehicle-treated rats.

The present invention is not intended to be limited to the foregoing example, but encompasses all such modifications and variations as come within the scope of the appended claims.

We claim:

1. A method for preserving renal medullary blood flow caused by a renal disorder in a human or non-human animal comprising the step of:
administering an agent selected from the group consisting of 20-HETE and a 20-HETE analog to the human or non-human animal in an amount sufficient to prevent to treat the renal disorder.

2. The method of claim 1, wherein the method is for preserving medullary blood flow in a human subject.

3. The method of claim 1, wherein the renal disorder is selected from the group consisting of proteinuria, diabetes-induced nephropathy, hypertension-induced nephropathy, kidney transplantation rejection, Heyman nephritis, remnant kidney nephropathy, ureteral obstruction nephropathy, a kidney disease caused by radiation, a kidney disease caused by an immunosuppressive drug, a kidney disease caused by a nephrotoxic drugs or toxins, renal ischemia-reperfusion injury and renal injury associate with shock.

4. The method of claim 1, wherein the renal disorder is renal ischemia-reperfusion injury and renal injury associate with shock.

5. The method of claim 1, wherein the agent is a 20-BETE analog.

6. The method of claim 5, wherein the 20-HETE analog is defined by the formula: 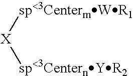
wherein R₁ is selected from the group consisting of carboxylic acid, phenol, amide, amine, imide, sulfonamide, sulfonamide, active methylene, 1,3-dicarbonyl, alcohol, thiol, tetrazole and other heteroaryl groups;
R₂ is selected from the group consisting of carboxylic acid, phenol, amine, amide, imide, sulfonamide, sulfonamide, active methylene, 1,3-dicarbonyl, alcohol, thiol, tetrazole and other heteroaryl groups;
W is a carbon chain (C₁ through C₂₅) and may be linear, cyclic, or branched and may comprise heteroatoms;
Y is a carbon chain (C₁ through C₂₅) and may be linear, cyclic, or branched and may comprise heteroatoms;
sp^{<3} Center is selected from the group consisting of vinyl, aryl, heteroaryl, cyclopropyl, and acetylenic moieties;
X is an alkyl chain that may be linear, branched, cyclic or polycyclic and may comprise heteroatoms;
m is 0, 1, 2, 3, 4 or 5; and
n is 0, 1, 2, 3, 4 or 5.

7. The method of claim 6, wherein the compound has a carboxyl or other ionizable group at either R₁ or R₂ and wherein the compound comprises a double bond or other functional group at a distance equal to 14-15 carbons from the ionizable group.

8. The method of claim 6, wherein the compound comprises a length of 20-21 carbons, has a carboxyl or other ionizable group at either R₁ or R₂, comprises a double bond or other functional group at a distance equal to 14-15 carbons from the ionizable group, and comprises a hydroxyl group on the 20 or 21 carbon at either R₁ or R₂.

9. The method of claim 5, wherein the 20-HETE analog is selected from the group consisting of 20-hydroxyeicosanoic acid, 20-hydroxyeicosa-5(Z),14(Z)-dienoic acid (WIT003), N-methyl sulfonyl-20-hydroxyeicosa-5(Z),14(Z)-dienamide 5(Z),14(Z) 20-hydroxyeicosadienoic acid (5,14-20-HEDE) and N-[20-hydroxyeicosa-5(Z),14(Z)-dienoyl]glycine (5, 14-20-HEDGE).

10. The method of claim 9, wherein the 20-HETE analog is N-[20-hydroxyeicosa-5(Z),14(Z)-dienoyl]glycine (5, 14-20-HEDGE).

11. The method of claim 1 further comprising the step of:
observing an improvement in renal medullary blood flow in the human or non-human animal.

12. The method of claim 11, wherein the improvement is an attenuation of a decrease in renal medullary blood flow brought about by the renal disease.

13. A method for treating ischemic acute renal failure in a human or non-human animal comprising the step of:
administering a 20-HETE analog selected from the group consisting of N-methylsulfonyl-20-hydroxyeicosa-5(Z), 14(Z)-dienamide 5(Z), 14(Z) 20-hydroxyeicosadienoic acid (5,14-20-HEDE) and N-[20-hydroxyeicosa-5(Z), 14(Z)-dienoyl]glycine (5, 14-20-HEDGE) to the human or non-human animal in an amount sufficient to prevent or treat ischemic acute renal failure.

14. The method of claim 13, wherein the method is for preventing or treating ischemic acute renal failure in a human.

15. The method of claim 13 further comprising the step of monitoring the improving post-ischemia medullary blood flow of the kidney.
